(19) United States
(12) Patent Application Publication
(10) Pub. No.: US 2008/0228146 A1
(43) Pub. Date: Sep. 18, 2008

(54) POSITIONING DEVICE FOR OSTIAL LESIONS

(76) Inventors: YOAV SHAKED, Tzoran (IL); Ronald J. Solar, San Diego, CA (US)

Correspondence Address: DANIEL J SWIRSKY, 55 REUVEN ST., BEIT SHEMESH, 99544, IL

(21) Appl. No.: 11/685,228

(22) Filed: Mar. 13, 2007

(51) Int. Cl. A61M 25/04

(52) U.S. Cl. 604/180

(57) ABSTRACT

A device and methods for positioning of a catheter at an ostium includes an anchoring portion and an expandable portion. The anchoring portion is configured to be attached to the shaft of the catheter at a location proximal to a treatment device positioned on the catheter. The expandable portion extends from a distal end of the anchoring portion and is immovable with respect to the anchoring portion along a longitudinal axis, The expandable portion is configured to extend outwardly with respect to the longitudinal axis and is configured to be bendable in a direction of a proximal end of the anchoring portion.

### FIELD OF THE INVENTION

The present invention relates to a positioning device for ostial lesions and, more particularly to a device that can be attached to a catheter to aid in positioning of the catheter at an ostial lesion.

### BACKGROUND OF THE INVENTION

Treating a lesion at an ostium is often challenging, due to the difficulty in positioning of a treatment device, such as a stent. Often, placement is too far within the branch vessel, preventing treatment of the lesion. At other times, placement is not far enough within the branch vessel, causing the stent to protrude into the main vessel. The aorta presents additional challenges since it is not visible during insertion of the treatment device.

Prior art devices designed for positioning stents in an ostial lesion are often undesirable since the positioning portion is incorporated within the delivery device or the stent, requiring the user to purchase the entire device. It would thus be beneficial to have a stand-alone device for positioning of any suitable delivery system in a ostial lesion.

An example of a stand-alone device is disclosed in U.S. Patent Application Publication No. 2005/0101968A to Dadourian. There is disclosed an ostial locator wire attached to a catheter wherein a selectively deployable expandable section of the ostial locator wire encircles the interventional device.

A disadvantage of the device disclosed in U.S. Patent Application Publication No. 2005/0101968A is that the ostial locator wire, when encircling the catheter, can become entangled with a stent placed thereon. Moreover, the wire is attached to the catheter via a sheath having a lumen and a fastener, which adds to the overall profile of the system.

### SUMMARY OF THE INVENTION

According to one aspect of the present invention, there is provided a stand-alone device for positioning of a catheter at an ostium. The device includes an anchoring portion configured to be directly attached to the catheter at a location proximal to a treatment device positioned on the catheter, the anchoring portion having a proximal end, a distal end and a longitudinal axis extending from the proximal end to the distal end, and an expandable portion extending from the distal end of the anchoring portion, the expandable portion immovable with respect to the anchoring portion along the longitudinal axis, the expandable portion configured to extend outwardly with respect to the longitudinal axis and configured to be bendable in a direction of the proximal end of the anchoring portion.

According to further aspects of the present invention, there is provided a method for positioning of a catheter in the ostium of a vessel branching off from a main vessel. The method includes providing a device having an anchoring portion and an expandable portion, the anchoring portion configured to be attached to a catheter shaft, and the expandable portion located at a distal end of the anchoring portion and configured to extend outwardly with respect to a longitudinal axis of the anchoring portion, providing a catheter having a shaft along its length and a treatment device at a distal end thereof, attaching the anchoring portion to the shaft of the catheter such that the anchoring portion and the expandable portion are positioned proximal to the treatment device, positioning a guiding catheter in a vessel to a location proximal to the ostium, placing a guidewire through the guiding catheter and into the ostium, advancing the catheter with the device attached thereto over the guidewire and through the guiding catheter, wherein the expandable portion is folded back proximally within the guiding catheter, advancing the catheter with the device attached thereto past a distal end of the guiding catheter, causing the expandable portion to be released from the guiding catheter and to extend outwardly with respect to the longitudinal axis of the anchoring portion, pushing the catheter into the ostial vessel until the expandable portion pushes against a wall of the main vessel at the ostium, preventing further advancement of the catheter, deploying the treatment device once the catheter is in position in the ostial vessel, and pulling the catheter and the attached treatment device proximally through the guiding catheter.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The above and further advantages of the present invention may be better understood by referring to the following description in conjunction with the accompanying drawings in which:

FIG. 1A is a schematic illustration of a device for positioning of a catheter in an ostial lesion;

FIG. 1B is a cross-sectional view of the device of FIG. 1A;

FIG. 2A is a schematic illustration of the device of FIG. 1A positioned on a catheter;

FIG. 2B is a cross-sectional view of the device of FIG. 2A;

FIG. 3A is a schematic illustration of the device of FIG. 1A having a double helix configuration and an expanded diameter in accordance with one embodiment of the present invention;

FIG. 3B is a schematic illustration of the device of FIG. 3A having a minimized diameter;

FIG. 4A is a schematic illustration of the device of FIG. 1A having a stent-like configuration and an expanded diameter in accordance with another embodiment of the present invention;

FIG. 4B is a schematic illustration of the device of FIG. 4A having a minimized diameter;

FIG. 5A is a schematic illustration of the device of FIG. 1A having a braided configuration and an expanded diameter in accordance with another embodiment of the present invention;

FIG. 5B is a schematic illustration of the device of FIG. 5A having a minimized diameter;

FIG. 6A is a schematic illustration of the device of FIG. 1A having a coil configuration and an expanded diameter in accordance with another embodiment of the present invention;

FIG. 6B is a schematic illustration of the device of FIG. 6A having a minimized diameter;

FIG. 7A is a schematic illustration of the device of FIG. 1A having a tube configuration and an expanded diameter in accordance with another embodiment of the present invention;

FIG. 7B is a schematic illustration of the device of FIG. 7A having a minimized diameter;

FIGS. 8A-8D are schematic illustrations of the device of FIG. 1A depicting an expandable portion in accordance with various embodiments of the present invention;

FIG. 9A is a schematic illustration of a mounting unit with a catheter placed therein;

FIG. 9B is a schematic illustration of the mounting unit of FIG. 9A and further including the device of FIG. 1A mounted thereon;

FIG. 10A is a schematic illustration of a guiding catheter to be used for introduction of the catheter and the device of the present invention into a vessel;

FIG. 10B is a schematic illustration of the guiding catheter of FIG. 10A with the catheter and device placed therein; and

FIGS. 11A-11E are schematic illustrations of a method of positioning of a catheter in an ostial lesion in accordance with embodiments of the present invention.

It will be appreciated that for simplicity and clarity of illustration, elements shown in the drawings have not necessarily been drawn accurately or to scale. For example, the dimensions of some of the elements may be exaggerated relative to other elements for clarity or several physical components may be included in one functional block or element. Further, where considered appropriate, reference numerals may be repeated among the drawings to indicate corresponding or analogous elements. Moreover, some of the blocks depicted in the drawings may be combined into a single function.

### DETAILED DESCRIPTION

In the following detailed description, numerous specific details are set forth in order to provide a thorough understanding of the present invention. It will be understood by those of ordinary skill in the art that the present invention may be practiced without these specific details. In other instances, well-known methods, procedures, components and structures may not have been described in detail so as not to obscure the present invention.

The present invention is directed to a positioning device for treatment of an ostial lesion. The principles and operation of a system and methods according to the present invention may be better understood with reference to the drawings and accompanying descriptions.

Before explaining at least one embodiment of the present invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

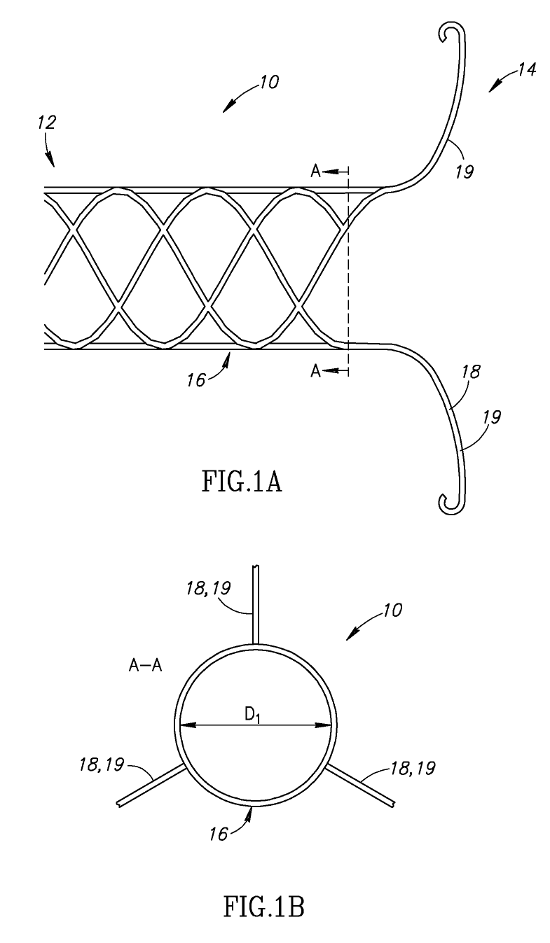

Reference is now made to FIG. 1A, which is a schematic illustration of a device 10 for positioning of a catheter in an ostial lesion. Device 10 has a proximal end 12 and a distal 14, and includes an anchoring portion 16 and an expandable portion 18. Anchoring portion 16 is configured to surround a catheter, which may be any available catheter off the shelf, and to be anchored to a shaft of the catheter. In some embodiments, anchoring portion 16 has a thickness of 0.002-0.050 inches and more preferably has a thickness in a range of 0.002-0.010 inches. The relatively low thickness is designed to keep the overall profile low, even when device 10 is placed on the catheter shaft. A length of anchoring portion 16 may vary from 1-10 cm, and more preferably from 3-5 cm. Referring to FIG. 1B, which is a cross-sectional view of device 10 along lines A-A, anchoring portion 10 has a first diameter D1 prior to anchoring. Expandable portion 18 is in some embodiments comprised of extension elements 19 which are pre-shaped to extend outwardly from anchoring portion 16, but which may be forcibly folded back in the direction of proximal end 12 during delivery. It is a feature of the present invention that extension elements 19 are configured in such a way so as to avoid entanglements with a stent positioned on the catheter. In embodiments of the present invention, extension elements 19 are 5-10 mm in length. Expandable portion 18 is configured such that it is immovable with respect to the anchoring portion along the longitudinal axis. This may be accomplished by manufacturing anchoring portion 16 and expandable portion 18 from one material. Alternatively, expandable portion 18 may be comprised of a different material than anchoring portion 16, and may be attached thereto via any suitable attachment means.

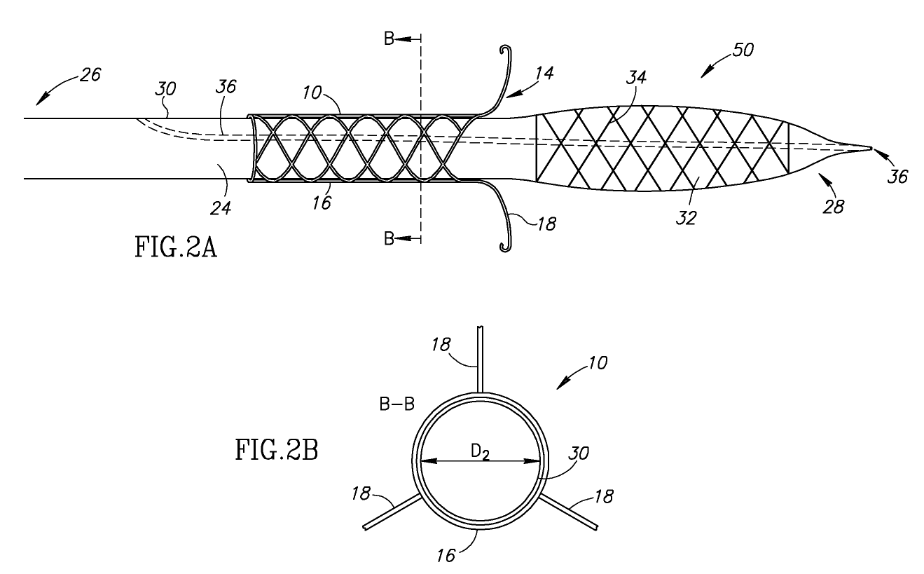

Reference is now made to FIGS. 2A and 2B, which are a schematic illustration of device 10 positioned on a catheter 24 and a cross-sectional view of device 10 positioned on catheter 24 along lines B-B, respectively. Catheter 24 has a proximal end 26, a distal end 28 and a shaft 30 running along its length. A treatment device 50 is positioned at distal end 28. In some embodiments, treatment device 50 is a balloon 32 positioned at distal end 28 and may optionally include a stent 34 positioned on balloon 32, as shown in FIG. 2A. In other embodiments, treatment device 50 is a self-expanding stent, a drug delivery mechanism, a cauterizing tip, or any other treatment device which can be used with a catheter. A guidewire lumen 36 runs through balloon 32 and at least a portion of shaft 30. Catheter 24 may be any catheter, such as an over-the-wire catheter, a rapid exchange catheter, or variations thereof. Alternatively, catheter 24 may be a catheter without a balloon or a stent, such as a laser catheter or any other catheter which may be used for treating a vessel. Device 10 is designed to be positionable on and anchored to any catheter or delivery system having a shaft. The catheter design depicted in the present application is for description purposes only and should not be regarded as limiting.

Device 10 is positionable on shaft 30 of catheter 24, and can be anchored thereto, as shown in FIG. 2A. When anchored onto shaft 30, anchoring portion 16 has a second diameter D2 as shown in FIG. 2B, wherein second diameter D2 is smaller than first diameter D1. Device 10 is positioned proximal to balloon 32 of catheter 24, such that distal end 14 including expandable portion 18 in its extended or folded back position also remains proximal to balloon 32. The exact position of device 10 on shaft 30 may be determined just prior to insertion of catheter 24 in a body, as will be described in further detail hereinbelow. It is a particular feature of the present invention that once device 10 is anchored onto shaft 30, it remains on shaft 30 for the entire duration of the procedure. Thus, when catheter 24 is removed from the body, device 10 is removed as well.

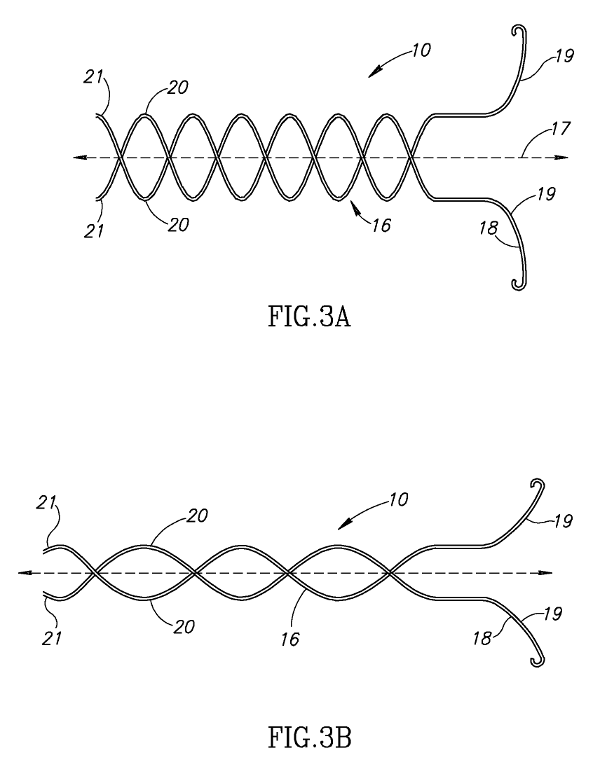

Anchoring portion 16 may be of various shapes and configurations. Reference is now made to FIGS. 3A and 3B, which are schematic illustrations of device 10 showing anchoring portion 16 comprised of a double helix, in an enlarged (pre-anchored) and minimized (anchored) state, respectively. As shown in FIG. 3A, two strands of wire 20 may be shaped in a double helix formation (similar to a DNA structure), wherein each wire 20 crosses over the other and both wires 20 are configured to surround shaft 30 of catheter 24. Proximal ends 21 of wires 20 may be pulled back proximally, causing anchoring portion 16 to contract to a minimized diameter and to contact shaft 30 of catheter 24. This contracted state provides anchoring of anchoring portion 16 to shaft 30. In some embodiments, a sticky coating is applied to a portion of wires 20 to ensure that anchoring is stable. Extension elements 19 of expandable portion 18 are formed from extensions of wires 20, and may be pre-formed in a bent configuration at an angle to a longitudinal axis 17 of anchoring portion 16. In some embodiments, the angle between extension elements 19 and longitudinal axis 17 of anchoring portion 16 is approximately 90 degrees. In other embodiments, the angle may vary from 60 degrees to 120 degrees. Although wires of expandable portion 18 are pre-formed at a particular angle, they may be forcibly folded back, for example, in a proximal direction so as to be positionable within a guiding catheter or a sheath, as will be described hereinbelow. The two configurations of extension elements 19—outward at an angle or folded proximally back—both ensure that extension elements 19 will not become entangled with stent 34.

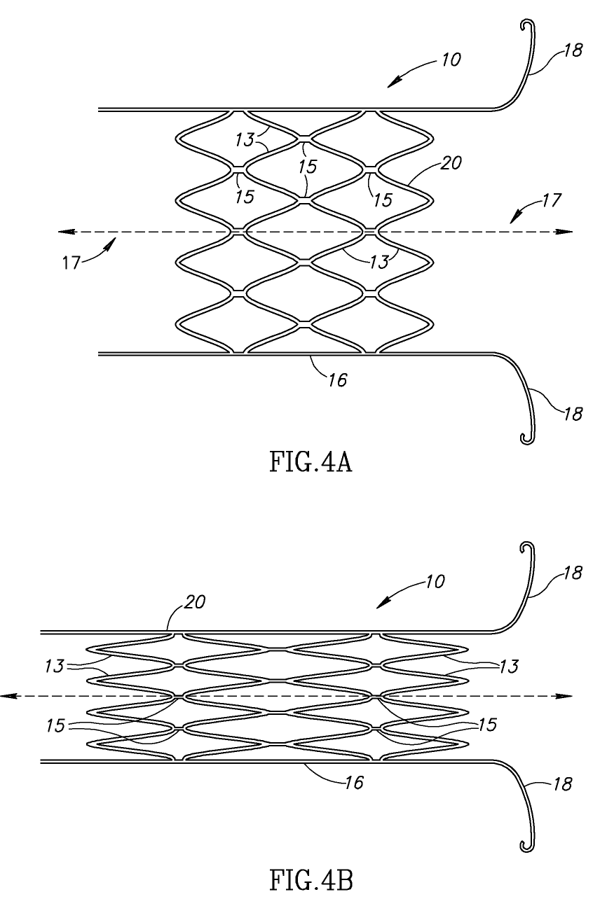

Reference is now made to FIGS. 4A and 4B, which are schematic illustrations of device 10 showing anchoring portion 16 comprised of a stent-like configuration, in an enlarged (pre-anchored) and minimized (anchored) state, respectively. As shown in FIG. 4A, a wire 20 may be shaped in a stent-like configuration, having struts 13 and connecting elements 15 forming a tube-like structure for example. It should be readily apparent that any stent or mesh-like configuration may be used. Anchoring portion 16 may be crimped onto shaft 30 of catheter 24, as is commonly done to actual stents when positioned on balloons. In the crimped, contracted state, anchoring portion 16 is anchored to shaft 30. In some embodiments, a sticky coating is applied to an internal portion of wires 20 to ensure that anchoring is stable. Extension elements 19 of expandable portion 18 are comprised of extensions of wires 20, and may be pre-formed in a bent configuration at an angle to longitudinal axis 17 of anchoring portion 16. In some embodiments, the angle between expandable portion and longitudinal axis 17 of anchoring portion 16 is approximately 90 degrees. In other embodiments, the angle may vary from 60 degrees to 120 degrees. Although wires of expandable portion 18 are pre-formed at a particular angle, they may be forcibly folded back, for example, in a proximal direction so as to be positionable within a guiding catheter or a sheath, as will be described hereinbelow. The two configurations of extension elements 19—outward at an angle or folded proximally back—both ensure that extension elements 19 will not become entangled with stent 34.

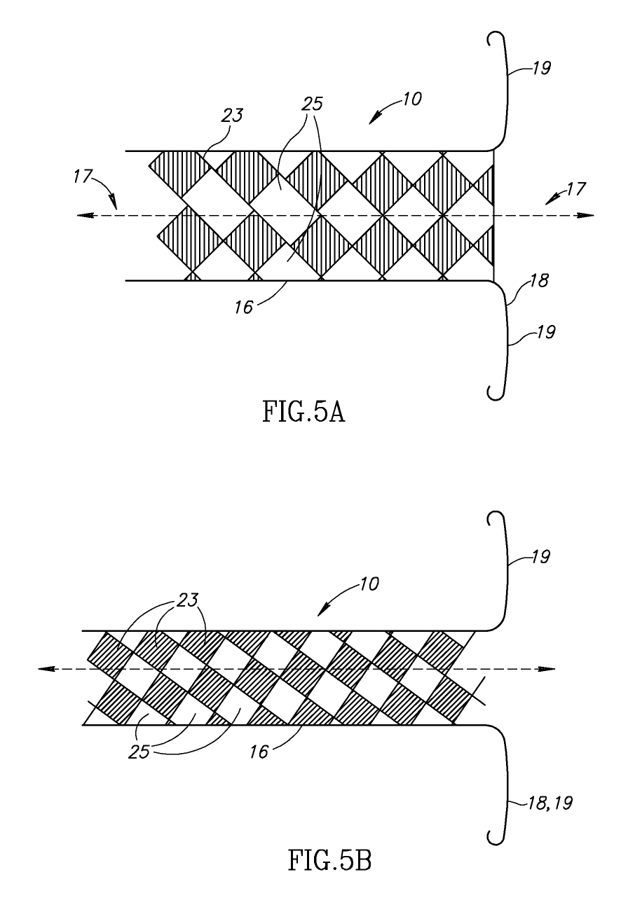

Reference is now made to FIGS. 5A and 5B, which are schematic illustrations of device 10 showing anchoring portion 16 in a braided configuration, in an enlarged (pre-anchored) and minimized (anchored) state, respectively. As shown in FIG. 5A, a series of strips may be shaped in a braided or woven configuration, wherein a first set of strips 23 passes over and under a second set of strips 25, and the second set of strips 25 passes over and under the first set of strips 23. This configuration may work similar to a Chinese finger trap, wherein upon pulling the ends outwardly, the diameter is reduced and anything caught in between is locked in. Thus, anchoring portion 16 may be anchored onto shaft 30 of catheter 24, by pulling one or both ends of anchoring portion outwardly after placement around shaft 30. In some embodiments, a sticky coating is applied to an internal portion of strips 23, 25 to ensure that anchoring is stable. Extension elements 19 of expandable portion 18 are formed from extensions of strips 23, 25, and may be pre-formed in a bent configuration at an angle to longitudinal axis 17 of anchoring portion 16. In some embodiments, the angle between expandable portion and longitudinal axis 17 of anchoring portion 16 is approximately 90 degrees. In other embodiments, the angle may vary from 60 degrees to 120 degrees. Although wires of expandable portion 18 are pre-formed at a particular angle, they may be forcibly folded back, for example, in a proximal direction so as to be positionable within a guiding catheter or a sheath, as will be described hereinbelow. The two configurations of extension elements 19—outward at an angle or folded proximally back—both ensure that extension elements 19 will not become entangled with stent 34.

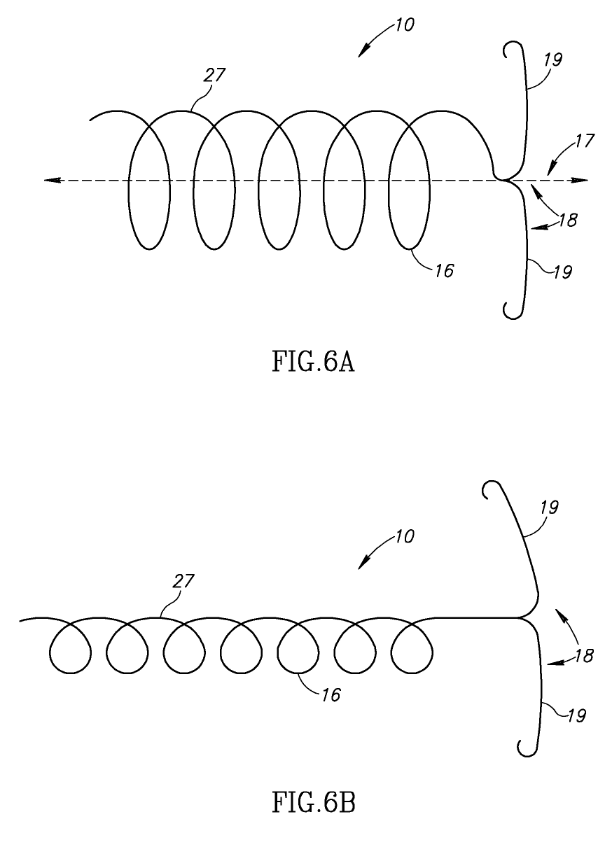

Reference is now made to FIGS. 6A and 6B, which are schematic illustrations of device 10 showing anchoring portion 16 comprised of a coil configuration, in an enlarged (pre-anchored) and minimized (anchored) state, respectively. As shown in FIG. 6A, a wire 27 is shaped into a coil configured to wrap around shaft 30. Anchoring portion 16 may be anchored onto shaft 30 of catheter 24, by pulling a proximal end of wire 27 proximally, thus reducing the diameter of anchoring portion 16 and causing the coil to contact shaft 30. In some embodiments, a sticky coating is applied to a portion of wire 27 to ensure that anchoring is stable. Extension elements 19 of expandable portion 18 are formed from an extension of wire 27 which may be split into one, two, three or more individual wire portions. Wires of expandable portion 18 may be pre-formed in a bent configuration at an angle to longitudinal axis 17 of anchoring portion 16. In some embodiments, the angle between expandable portion and longitudinal axis 17 of anchoring portion 16 is approximately 90 degrees. In other embodiments, the angle may vary from 60 degrees to 120 degrees. Although wires of expandable portion 18 are pre-formed at a particular angle, they may be forcibly folded back, for example, in a proximal direction so as to be positionable within a guiding catheter or a sheath, as will be described hereinbelow. The two configurations of extension elements 19—outward at an angle or folded proximally back—both ensure that extension elements 19 will not become entangled with stent 34.

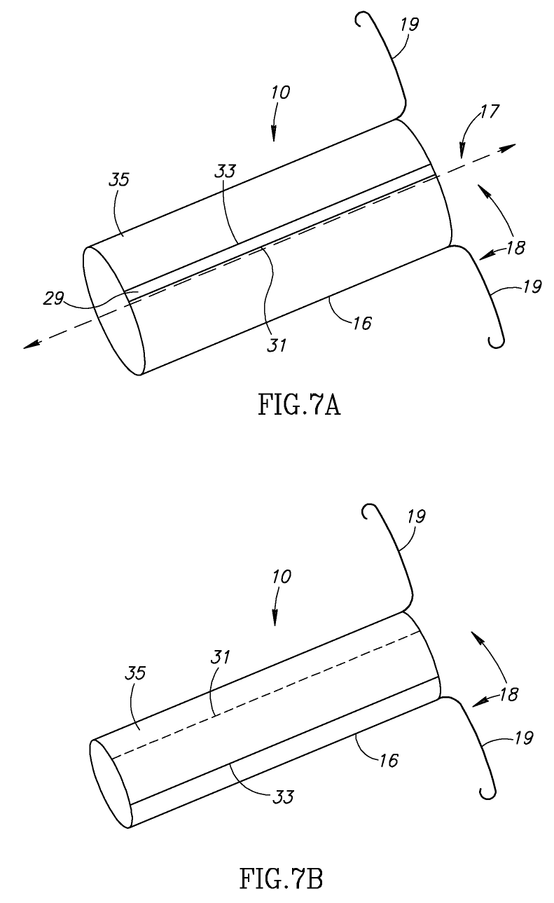

Reference is now made to FIGS. 7A and 7B, which are schematic illustrations of device 10 showing anchoring portion 16 comprised of a tube, in an enlarged (pre-anchored) and minimized (anchored) state, respectively. As shown in FIG. 7A, a tube 35 has a seam 29 which connects a first edge 31 and a second edge 33. In some embodiments, first edge 31 and second edge 33 are not connected to each other. Anchoring portion 16 may be anchored onto shaft 30 of catheter 24, by folding second edge 33 over first edge 31 as shown in FIG. 7B, or vice versa. A locking mechanism locks the overlapping positioning of first and second edges 31 and 33. The locking mechanism may be, for example, a glue. Alternatively, the locking mechanism may be a clam shell or any other method of attaching one side of a tube to another. In some embodiments, a sticky coating is applied to an inner portion of tube 35 to ensure that anchoring is stable. Extension elements 19 of expandable portion 18 are comprised of portions extending from tube 35, and may be pre-formed in a bent configuration at an angle to longitudinal axis 17 of anchoring portion 16. In some embodiments, the angle between expandable portion and longitudinal axis 17 of anchoring portion 16 is approximately 90 degrees. In other embodiments, the angle may vary from 60 degrees to 120 degrees. Although wires of expandable portion 18 are pre-formed at a particular angle, they may be forcibly folded back, for example, in a proximal direction so as to be positionable within a guiding catheter or a sheath, as will be described hereinbelow. The two configurations of extension elements 19—outward at an angle or folded proximally back—both ensure that extension elements 19 will not become entangled with stent 34.

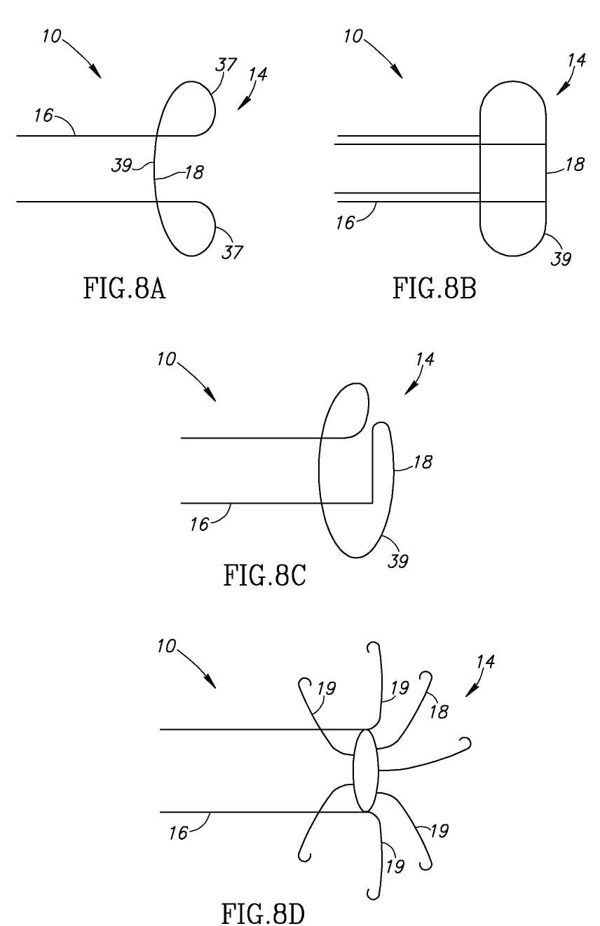

Reference is now made to FIGS. 8A-8D, which are schematic illustrations showing distal end 14 of device 10, depicting expandable portion 18 in accordance with several additional embodiments of the present invention. These depictions are exemplary and should not be regarded as limiting. In a first embodiment, as shown in FIG. 8A, two extension portions 37 similar to extension elements 19 of FIG. 1A are positioned at distal end 14, and are connected to each other via a connecting element 39. In another embodiment, as shown in FIG. 8B, connecting element 39 forms a complete circle, wherein expandable portion 18 may include two or more extension portions 37, and wherein all of extension portions 37 are connected to each other via connecting element 39. In another embodiment, shown in FIG. 8C, connecting element 39 forms a spiral configuration. In yet another embodiment, shown in FIG. 8D, multiple extension elements 19 are used. The number of extension elements 19 may vary from 2 to any number depending on the dimensions of extension elements 19. It should be readily apparent that many other configurations are possible.

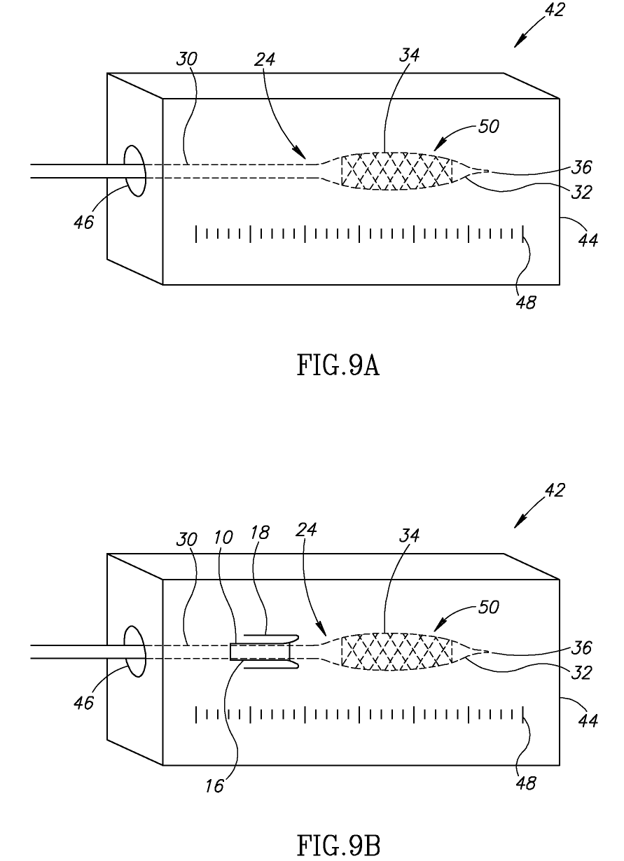

Reference is now made to FIGS. 9A and 9B, which are schematic illustrations of a mounting unit 42 used for mounting device 10 onto catheter 24, which may include any catheter off the shelf. Mounting unit 42 includes a transparent box 44 having an access port 46 and measurement units 48 included thereon. Mounting unit 42 is comprised of a material which can be sterilized, such that the entire mounting procedure may be performed under sterile conditions. Catheter 24 is placed through access port 46 and into box 44, as shown in FIG. 9A. Device 10 is then introduced through access port 46, and positioned on shaft 30 in accordance with measurements determined from measurement units 48. Thus, a physician may decide on a case-by-case basis how far proximally or distally to position device 10 with respect to treatment device 50. This decision may be based on anatomical considerations, or there may be standard measurements which can be used. In some embodiments, device 10 is positioned just proximal to treatment device 50. In other embodiments, device 10 is positioned several millimeters proximal to treatment device 50. Any position may be chosen. Once the optimal position is determined, device 10 is anchored onto shaft 30 in accordance with the methods described above.

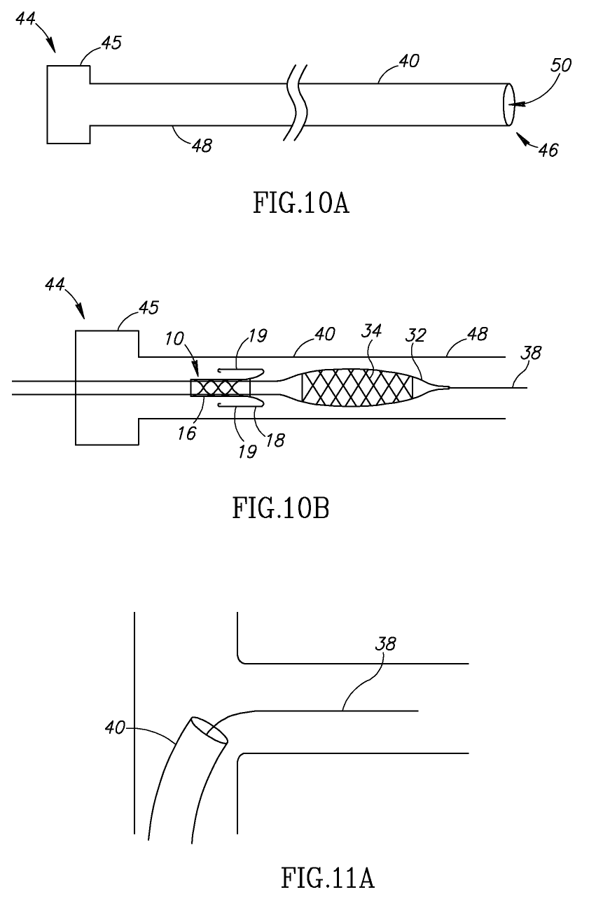

In embodiments of the present invention, catheter 24 with device 10 anchored thereon is introduced into the vessel using a guiding catheter 40, as shown in FIGS. 10A and 10B. A standard guiding catheter 40, as shown in FIG. 10A, includes a proximal end 44, a distal end 46 and a shaft 48 connecting proximal end 44 and distal end 46. Shaft 48 defines a lumen 50 running therethrough, into which may be inserted any items which may be necessary to insert into a vessel, such as a guidewire or a catheter or any other delivery device. A hub 45 at proximal end 44 allows for insertion of such items into guiding catheter 40. As shown in FIG. 10B, when catheter 24 with device 10 anchored thereon is placed within guiding catheter 40, extension elements 19 of expandable portion 18 are folded back proximally so as to avoid entanglement with stent 34.

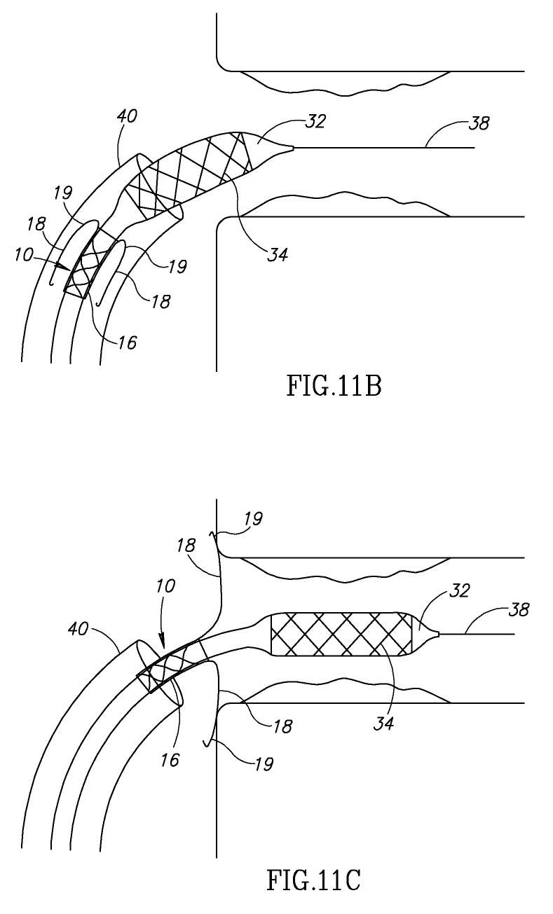

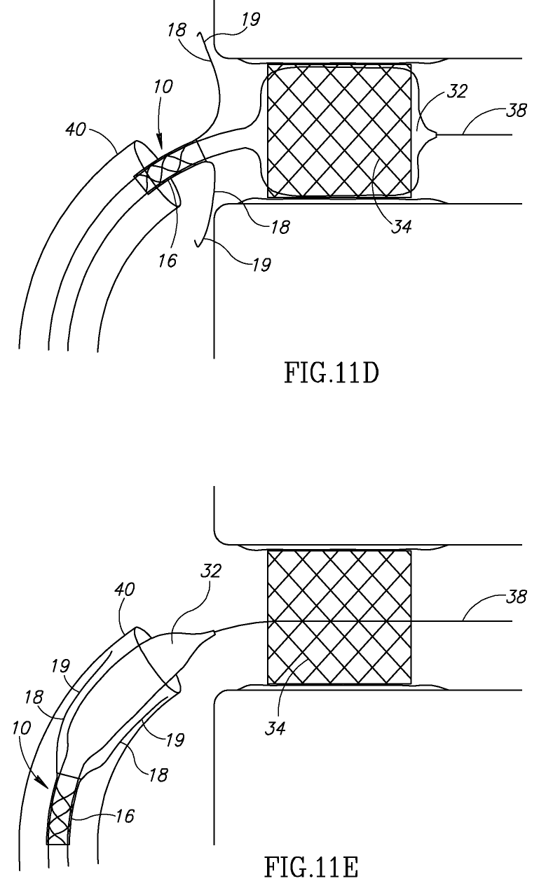

Reference is now made to FIGS. 11A-11E, which are schematic illustrations showing a method of using device 10 in accordance with embodiments of the present invention. Although the method is described with respect to a balloon catheter, it should be readily apparent that any catheter having any type of treatment device thereon may be used. First, a guidewire 38 is introduced into the vessel through guiding catheter 40, as shown in FIG. 11A. Next, catheter 24 with device 10 attached thereto is introduced over guidewire 38 and through guiding catheter 40, and guiding catheter 40 is advanced until it reaches a point proximal to the ostium of the vessel, as shown in FIG. 11B. Catheter 24 with device 10 anchored thereto is pushed distally, until extension elements 19 of expandable portion 18 of device 10 are released from guiding catheter 40, as shown in FIG. 11C. Extension elements 19 assume their pre-shaped configuration, extending outwardly from anchoring portion 16 and from shaft 30 of catheter 24. This outward extension causes extension elements 19 to contact the walls of the main vessel at the ostium, and they may be used as stoppers to prevent catheter 24 from advancing beyond that point, preventing an overly distal positioning of stent 34. Furthermore, they allow the user to advance catheter 24 until it reaches resistance from extension elements 19 pushing against the walls of the main vessel at the ostium. This prevents stent 34 from being positioned too far proximally and thus jutting out into the main vessel. Once catheter 24 is in place, balloon 32 can be expanded, thus expanding stent 34, as shown in FIG. 11D. The proper positioning by extension elements of expandable portion 18 of device 10 allows for the stent to be positioned properly in the vessel. Finally, catheter 24 with device 10 still attached thereto is pulled proximally into guiding catheter 40, which is then removed from the vessel, as shown in FIG. 11E. When catheter 24 is pulled proximally into guiding catheter 40, extension elements 19 of expandable portion 18 may be folded forward distally, since it is no longer important to avoid entanglement with stent 34.

Device 10 may be made from any biocompatible material, such as metals, polymers or combinations thereof. In some embodiments, at least expandable portion 18 is comprised of a shape-memory alloy or super-elastic material such as Nitinol™.

While certain features of the present invention have been illustrated and described herein, many modifications, substitutions, changes, and equivalents may occur to those of ordinary skill in the art. For example, a catheter for uses other than expansion of a balloon and/or delivery of a stent may be used with the device of the present invention, such as a catheter for drug delivery at an ostium, for cauterization, or for any other treatment. It is, therefore, to be understood that the appended claims are intended to cover all such modifications and changes as fall within the true spirit of the present invention.

What is claimed is:

1. A stand-alone device for positioning of a catheter at an ostium, the device comprising:
an anchoring portion configured to be directly attached to the catheter at a location proximal to a treatment device positioned on the catheter, said anchoring portion having a proximal end, a distal end, and a longitudinal axis extending from said proximal end to said distal end; and
an expandable portion extending from said distal end of said anchoring portion, said expandable portion immovable with respect to said anchoring portion along the longitudinal axis said expandable portion configured to extend outwardly with respect to said longitudinal axis and configured to be bendable in a direction of said proximal end of said anchoring portion.

2. The device of claim 1, wherein said anchoring portion comprises a wire mesh.

3. The device of claim 1, wherein said anchoring portion comprises a coil.

4. The device of claim 1, wherein said anchoring portion comprises a double helix.

5. The device of claim 1, wherein said anchoring portion comprises a stent-like configuration.

6. The device of claim 1, wherein said anchoring portion comprises a braid.

7. The device of claim 1, wherein said anchoring portion comprises a tube.

8. The device of claim 1, wherein said anchoring portion comprises a first diameter prior to attachment to the catheter, and a second diameter upon attachment to said catheter, wherein said second diameter is smaller than said first diameter.

9. The device of claim 8, wherein said anchoring portion is configured to be attached to the catheter by changing said first diameter to said second diameter.

10. The device of claim 1, wherein said anchoring portion is configured to be attached to the catheter by a clam shell.

11. The device of claim 1, wherein said anchoring portion is configured to be attached to the catheter by a sticky coating.

12. The device of claim 1, wherein said anchoring portion is configured to at least partially surround a shaft of the catheter.

13. The device of claim 12, wherein said anchoring portion is configured to fully surround a shaft of the catheter.

14. The device of claim 1, wherein said anchoring portion is configured to be permanently attached to the catheter.

15. The device of claim 1, wherein said expandable portion comprises multiple extension elements.

16. The device of claim 15, wherein said extension elements are connected to each other by a connecting element.

17. The device of claim 1, wherein said expandable portion is comprised of a shape-memory alloy.

18. The device of claim 1, wherein said anchoring portion has a thickness of between 0.002 inches and 0.010 inches.

19. The device of claim 1, wherein the catheter is a balloon catheter.

20. A method for positioning of a catheter in the ostium of a vessel branching off from a main vessel, the method comprising:
providing a device having an anchoring portion and an expandable portion, said anchoring portion configured to be attached to a catheter shaft, and said expandable portion located at a distal end of said anchoring portion and configured to extend outwardly with respect to a longitudinal axis of said anchoring portion;
providing a catheter having a shaft along its length and a treatment device at a distal end thereof;
attaching said anchoring portion to said shaft of said catheter such that said anchoring portion and said expandable portion are positioned proximal to said treatment device;
positioning a guiding catheter in a vessel to a location proximal to the ostium;
placing a guidewire through said guiding catheter and into the ostium;
advancing said catheter with said device attached thereto over said guidewire and through said guiding catheter, wherein said expandable portion is folded back proximally within said guiding catheter;
advancing said catheter with said device attached thereto past a distal end of said guiding catheter, causing said expandable portion to be released from said guiding catheter and to extend outwardly with respect to said longitudinal axis of said anchoring portion;
pushing said catheter into the ostial vessel until said expandable portion pushes against a wall of the main vessel at the ostium, preventing further advancement of said catheter;
deploying said treatment device once said catheter is in position in the ostial vessel; and
pulling said catheter and said attached treatment device proximally through said guiding catheter.

21. The method of claim 20, wherein said catheter further includes a balloon and wherein said deploying said treatment device comprises expanding said balloon.

22. The method of claim 21, wherein said catheter further includes a stent, and wherein upon said expanding of said balloon, said stent is configured to expand.

23. The method of claim 20, wherein the main vessel is an aorta.

24. The method of claim 20, wherein said attaching is done by placing said catheter in a mounting unit having measurement units and further placing said device into said mounting unit and over said catheter at a location determined by said measurement units.

25. The method of claim 20, wherein said attaching is done by placing said device over a shaft of said catheter, and by minimizing a diameter of said device.

26. The method of claim 25, wherein said attaching is further done by placing a sticky coating on said anchoring portion of said device.
